(19) United States
(12) Patent Application Publication
(10) Pub. No.: US 2005/0214347 A1
(43) Pub. Date: Sep. 29, 2005

(54) Low-energy-diet

(76) Inventors: Arne Astrup, Klampenborg (DK); Soren Toubro, Vipperod (DK)

Correspondence Address: GIFFORD, KRASS, GROH, SPRINKLE & CITKOWSKI, P.C, PO BOX 7021, TROY, MI, 48007-7021, US

(21) Appl. No.: 10/518,993

(22) PCT Filed: Jun. 23, 2003
(86) PCT No.: PCT/DK03/00424

§ 371 Date: Dec. 21, 2004

Related U.S. Application Data
(60) Provisional application No. 60/402,529, filed on Aug. 12, 2002.

(30) Foreign Application Priority Data
Jun. 21, 2002 (DK) PA 2002 00959

(51) Int. Cl. A61K 47/00

(52) U.S. Cl. 424/439; 426/635

(57) ABSTRACT

The invention relates to a low-energy-diet (LED) having effect on the basal metabolic rate, the protein metabolism and/or the energy expenditure, such as a low-energy-diet comprising Carbohydrate: 70-120 g, Protein: 50-80 g, Fat: 5-20 g, including medium chain triglycerides, and Iron: 20-30 mg/d, wherein the carbohydrate source and/or the fat source may increase the energy expenditure.

### FIELD OF INVENTION

The present invention relates to a low-energy diet (LED) having effect on the basal metabolic rate, the protein metabolism and/or the energy expenditure.

All patent and non-patent references cited in the application, or in the present application, are also hereby incorporated by reference in their entirety.

### BACKGROUND OF INVENTION

Low-energy-diets are known from the prior art, for example EP 425 423 presents a low-energy-diet wherein specific fat, carbohydrate and protein sources are used. However, the low-energy-diets known either simply offer fewer calories than normal daily cost, or focus on one mechanism of losing weight.

### SUMMARY OF INVENTION

The present invention relates to a low-energy-diet having several improvements in relation to prior art diets.

The present diet is capable of increasing satiety yet providing weight loss. Furthermore, the diet maintains fat-free body mass, increases the energy expenditure, reduces or eliminates risk of iron deficiency, as well as magnesium and calcium deficiency.

In a preferred embodiment the diet also prevents heart arrhythmias and gall stones.

Accordingly, the present invention relates to a low-energy-diet comprising as ingredients sources of
protein, carbohydrate, fat, and optionally minerals and pH-regulating agents, wherein one or more of the ingredient(s) has effect on at least two of the following mechanisms
the basal metabolic rate, and/or
the protein metabolism, and/or
the energy expenditure,
when the low-energy-diet is administered as the main or sole nutrition.

The low-energy-diet is in particular intended for use as the main or sole nutrition in the treatment of overweight, by for example replacing one or more, or all of the daily meals of an individual being obese. But the diet may of course also be used as a nutritional supplement.

In another aspect the invention relates to a method for treating overweight comprising administering to an individual in need thereof an effective amount of a low-energy-diet as defined above as the main or sole nutrition daily, in particular the low-energy-diet is suitable for treatment of severe overweight also called obesity.

In the present context “overweight” is defined as a Body mass Index>25 kg/m². “Severe overweight” is defined as a body mass Index>30 kg/m².

Furthermore, the invention relates to the use of ingredients being sources of
protein, carbohydrate, fat, and optionally minerals and pH-regulating agents, wherein one or more of the ingredient(s) has effect on at least two of the following mechanisms
the basal metabolic rate, and/or
the protein metabolism, and/or
the energy utilization,
for the production of a low-energy-diet for administration as the main or sole nutrition.

### DRAWINGS

FIG. 1. Least Squares Means (SE) body weight of subjects during weight reduction by either high (black triangle, n=19) or low physical activity (open triangle, n=22), and the overall mean values (- - -, n=41). There were no differences between interventions, thus a significant effect of time (trend, P<0.0001), with an overall slope β(95% confidence limits): β=−1.08 kg/week (−1.22-0.95).

FIG. 2. Percentage weight loss during 7 weeks intervention using Speasy® as the only nutrient. Values are Least Squares Means (95% confidence limits - - -)) n=41 observations. Trend P<0.0001; Slope β (95% confidence limit): β=1.186% weight loss/week (1.063-1.309).

### DETAILED DESCRIPTION OF THE INVENTION

Definitions

Daily amounts: In the present context the amounts of ingredients in the low-energy-diet is given as daily doses, i.e. for example as mg/d. Since the low-energy-diet may be divided into two or more doses per day, the amount of the various ingredients are calculated on basis of the total low-energy-diet per day.

It is an object of the present invention to provide a diet having an improved profile as compared to prior art diets in respect of at least the following parameters:
satiety
weight loss
maintaining fat-free body mass
increase in energy expenditure

This is obtained by using ingredients in the low-energy-diet that is capable of, preferably in a synergistic manner, to have effect on at least two of the following mechanisms
the basal metabolic rate, and/or
the protein metabolism, and/or
the energy expenditure,
   when the low-energy-diet is administered as the main or sole nutrition.

In a preferred embodiment the low-energy-diet is capable of having effect on all three of the following mechanisms the basal metabolic rate,
the protein metabolism,
the energy expenditure,
   when the low-energy-diet is administered as the main or sole nutrition.

Low-energy-diets administered to treat overweight are often taken for a long period of time, and therefore it is of importance that basal metabolic rate as well as protein metabolism is only mildly affected by the diet, if at all effected, so that the metabolism is stabilised although an individual to lose weight administer the low-energy-diet as main or sole nutrition for weeks or months. Thus, it is preferred that the ingredients having effect on the basal metabolic rate are capable of inhibiting the basal metabolic rate from decreasing. The ingredients having effect on the protein metabolism are preferably capable of reducing protein degradation.

Furthermore, the ingredients having effect on energy expenditure are preferably capable of increasing the energy expenditure.

Energy Content

In any diet form aiming at promoting weight loss, the diet is a balance between offering sufficiently low amount of energy and at the same time offering satiety sufficiently high to maintain the individual losing weight to follow the instructions of the diet.

In the present invention, the diet preferably has an energy content in the range of from 600 kcal/d to 1200 kcal/d, such as an energy content of approximately 800 kcal/d.

Basal Metabolic Rate

During a diet, in particular a low-energy-diet, the normal response in the body, is to lower the basal metabolic rate, due to the lower level of energy supply. However, when aiming at losing weight this normal reduction in the basal metabolic rate counteracts to some extent the effect of less energy intake. Accordingly, it is an object of the invention to prevent reduction in the basal metabolic rate.

In one embodiment this is accomplished by providing an iron content in the low-energy-diet, wherein said iron content is sufficient to prevent reduction in the basal metabolic rate, as discussed below.

Basal metabolic rate may be measured by indirect calometry either by a ventilated hood system or by use of respiration chambers.

Protein Metabolism

Normal metabolic responses to low-energy-diet are ketosis followed by acidosis leading to protein loss. According to the present invention, it is preferred to supply proteins in the diet, and also to supply with an ingredient capable of reducing or eliminating the protein loss, normally seen in individuals subjected to low-energy-diets.

The effect of an ingredient on the protein metabolism may be measured as the effect on the nitrogen metabolism, for example as described in Stanko et al. “Body composition, energy utilization, and nitrogen metabolism with a severely restricted diet supplemented with dihydroxyacetone and pyruvate”, Am J Clin Nutr (1992) 55, 771-6, hereby incorporated by reference.

The ingredient capable of reducing or eliminating the protein loss, preferably is an ingredient capable of reducing the acidosis following ketosis, thereby reducing the protein degradation, and as effect affecting the protein metabolism.

pH Regulation

One such protein metabolism effecting ingredient is this a pH-regulating ingredient. Accordingly, the low-energy-diet according to the invention preferably comprises a pH-regulating agent in an amount sufficient to reduce protein degradation. In a preferred embodiment the pH-regulating agent is bicarbonate in an amount sufficient to reduce protein degradation, such as bicarbonate in an amount of from 50 mmol/d to 70 mmol/d.

Protein

The protein content of the low-energy diet is preferably given in the amounts recommended by the health authorities. The protein source is preferably a source of proteins having a great bio-availability.

Thus, the source of protein is preferably selected from casein, pork protein, and/or soy protein. In a preferred embodiment at least a portion of the source of protein is soy protein.

In the low-energy-diet having a energy content of about 600 kcal/d to 1200 kcal/d the protein content is preferably in an amount of from 60 g protein to 75 g protein, such as about 70 g protein.

Energy Expenditure

Daily energy expenditure may be measured by indirect calorimetry during stay in a respiratory chamber as described in Dulloo et al. “Twenty-four-hour energy expenditure and urinary catecholamines of humans consuming low-to-moderate amounts of medium-chain triglycerides: a dose-response study in a human respiratory chamber, European Journal of Clinical Nutrition (1996) 50, 152-158, hereby incorporated by reference.

In order to promote the desired weight loss in individuals subjected to the low-energy diet, the low-energy-diet preferably also comprises at least one ingredient capable of increasing the energy expenditure.

This ingredient may be selected from one or more of the following:

Dihydroxy acetone phosphate, pyruvate, as well as medium chain triglycerides.

Accordingly, the low-energy-diet according to the invention preferably comprises as at least a part of the carbohydrate source dihydroxy acetone phosphate and/or pyruvate.

The dihydroxy acetone phosphate and/or pyruvate is preferably in an amount sufficient to increase the energy expenditure whether administered together or alone. Therefore, the diet preferably comprises dihydroxy acetone phosphate in an amount of from 5 g to 15 g, such as in an amount of from 10 g to 15 g. In another embodiment the diet preferably comprises pyruvate in an amount of from 5 g to 20 g, such as in an amount of from 10 g to 20 g.

The same amounts of dihydroxy acetone phosphate and pyruvate may also be administered when the two ingredients both are administered in the low-energy-diet.

The pyruvate preferably is in the form of sodium pyruvate and/or calcium pyruvate, for example as about 10 g of sodium pyruvate and 9 g of calcium pyruvate.

Carbohydrate

In addition to the carbohydrates given as pyruvate and dihydroxy acetone phosphate other sources of carbohydrate are also included in the low-energy-diet. Any suitable carbohydrate source may be used, for example fructose.

The low-energy-diet according to the invention preferably comprises the total carbohydrate source in an amount of from 70 g carbohydrate to 120 g carbohydrate, more preferably in an amount of from 80 g carbohydrate to 110 g carbohydrate.

Fat

Another ingredient capable of promoting the energy expenditure is medium chain triglycerides as discussed above, said medium chain triglycerides preferably being selected from C6 triglycerides, C8 triglycerides and C10 triglycerides.

Of the preferred amount of fat source in the low-energy-diet according to the invention in an amount of from 5 g to 20 g, the medium chain triglycerides preferably constitute at least 3 g of the fat source.

The remaining fat in the fat source is preferably unsaturated fatty acids, such as oleic acid.

Also, the low-energy-diet may comprise fish oil, such as at least 1 g of fish oil, such as at least 3 g of fish oil, such as at least 5 g of fish oil.

Iron Content

In a preferred embodiment the low-energy-diet according to the invention comprises iron in an amount sufficient to modulate the basal metabolic rate, such as sufficient to prevent reduction in the basal metabolic rate. In Beard et al. “Changes in iron status during weight loss with very-low-energy diets” Am J Clin Nutr (1997) 66, 104-10 the effect of iron intake during weight loss is described. The saturation of transferrin is increased when the iron intake is increased to above the recommended daily allowance (RDA) leading to a reduced decrease in the thyroid hormone T₃ level. Thereby the reduction in basal metabolic rate normally seen in individuals during a diet, in particular a low-energy-diet is reduced or even eliminated.

It is preferred that at least 2 mg/d iron above RDA should be included in the low-energy-diet, such as at least 5 mg/day iron above RDA. Most preferably about 9 mg/d iron above RDA should be included in the low-energy-diet. In other words, the low-energy-diet according to the invention preferably comprises iron in an amount of from 20 mg/d to 30 mg/d.

Satiety

Apart from stabilising the basal metabolic rate and the protein metabolism and increasing the energy expenditure, the low-energy-diet should preferably also provide the individual subjected to the low-energy-diet with a feeling of satiety each time the doses of low-energy-diet is taken. Thereby the probability of success of losing weight rapidly increases.

The ingredients, dihydroxy acetone phosphate, pyruvate and medium chain triglycerides, capable of increasing the energy expenditure may additionally have the effect of providing satiety.

Dietary Fibres

In addition to the ingredients mentioned above, the low-energy-diet according to the invention may further comprise dietary fibres, among others also to increase the feeling of satiety.

The dietary fibres may be any suitable dietary fibres, such as fibres are selected from barley fibres, sugar beat fibres, and oat fibres or combinations thereof.

Independent of the fibre used, the amount of fibre is preferably in an amount of from 10 g/d to 50 g/d.

Magnesium

In order to avoid disturbances in the electrolytes leading to heart arrhythmias it is further preferred that the diet comprises magnesium, such as magnesium added as MgCl, preferably in an amount of from 15 mEq/d to 20 mEq/d, preferably about 18 mEq/d.

Ursodeoxy Cholic Acid

The changes in the organism of an individual losing weight by means of a low-energy-diet may lead to side effects such as gall stones. In a preferred embodiment the diet further comprises ursodeoxy cholic acid to reduce the risk of gall stones. In another embodiment ursodeoxy cholic acid is administered separately as a tablet. In any event the ursodeoxy cholic acid is preferably administered in an amount of from 1000 mg/d to 1400 mg/d, preferably about 1200 mg/d.

Vitamins and Minerals

Vitamins and minerals not mentioned above are dosed within the limits laid down by the health authorities, and may be included in the low-energy-diet or administered daily in tablet form. In particular vitamins and minerals are dosed according to Dietary Reference Intakes, Institute of medicine, USA, with modifications according to special requirements for overweight subjects on a low-calorie diet.

In a preferred embodiment the low-energy-diet according to the invention comprises per day
Carbohydrate: 70-120 g, including dihydroxy acetone phosphate 10-15 g and/or pyruvate 10-20 g
Protein: 50-80 g
Fat: 5-20 g, including at least 3 g medium chain triglycerides
Iron: 20-30 mg/d

In another embodiment the low-energy-diet according to the invention comprises per day
Carbohydrate: 70-120 g,
Protein: 50-80 g
Fat: 5-20 g, including at least 4 g medium chain triglycerides
Iron: 20-30 mg/d

In a more preferred embodiment, the low-energy-diet according to the invention comprises per day
Carbohydrate: 70-120 g, including dihydroxy acetone phosphate 10-15 g and/or pyruvate 10-20 g
Protein: 50-80 g
Fat: 5-20 g, including at least 3 g medium chain triglycerides
Iron: 20-30 mg/d
Magnesium: 15-20 mEq/d

In an even more preferred embodiment the low-energy-diet according to the invention comprises per day:
Carbohydrate: 70-120 g, including dihydroxy acetone phosphate 10-15 g and/or pyruvate 10-20 g
Protein: 50-80 g
Fat: 5-20 g, including at least 3 g medium chain triglycerides
Iron: 20-30 mg/d
Magnesium: 15-20 mEq/d
Fibres: 10-50 g/d
   Formulation

The low-energy-diet according to the present invention may be formulated into any suitable form, such as powders, or ready-to-drink or ready-to-eat products. Thereby the individuals desiring to lose weight may adapt more to the diet, since they have a choice between various products.

In one embodiment the low-energy-diet is in the form of powder. Said powder may be dispensed in separate doses and suspended or solved in liquid, such as liquid selected from water and milk immediately before intake.

In another embodiment the low-energy-diet is formulated as a ready-to-drink product.

In yet another embodiment the low-energy-diet is formulated as a bar.

Applications

The low-energy-diet according to the invention is in particular meant for use in the treatment of overweight. Accordingly, the present invention further relates to a method for treating overweight comprising administering to an individual in need thereof an effective amount of a low-energy-diet as defined above as the main or sole nutrition daily.

The diet is preferably divided into two or more portions per day to be administered at regular times for, for example to simulate the traditional meal times.

Another aspect of the invention relates to the use of ingredients being sources of
protein, carbohydrate, fat, and optionally minerals and pH-regulating agents, wherein one or more of the ingredient(s) has effect on at least two of the following mechanisms
the basal metabolic rate, and/or
the protein metabolism, and/or
the energy expenditure,
for the production of a low-energy-diet for administration as the main or sole nutrition.

The ingredients mentioned may be as described above.

The diet is preferably packed in a package intended for being able to cover the total nourishment requirement for a defined period of time, such as packages for a powder, a ready-to-drink product or a bar product.

### EXAMPLES

### Example 1

Powder Formulation

A daily dose of the low-energy-diet
Carbohydrate: 96 g, including pyruvate 10 g
Protein (soy and casein): 70 g
Fat (oleic acid): 15 g
Fibres (barley): 15 g/d
Vitamins and minerals according to Nordis Alimantaris
Plus extra addition of iron: 16 g
Flavours: 3 g

### Example 2

Ready-to-Drink Formulation with Vanilla Flavour

| Formula | % |
|---|---|
| Water | 74.8 |
| Corn Syrup Solids, 24 | 8.2 |
| Carbohydrate including pyruvate (10% of total) | 6.8 |
| Protein (casein and soy 50/50) | 7.22 |
| Oil (oleic acid) | 1.146 |
| Lecithin | 0.4 |
| Dicalcium phosphate | 0.3 |
| Avicel, FMC CL611 | 0.241 |
| Vanilla flavours | 0.460 |
| Ca citrate | 0.134 |
| Mono potassium phosphate | 0.096 |
| Butterscotch | 0.05 |
| WSV premix | 0.048 |
| Viscarin | 0.02 |
| OSV premix | 0.019 |
| | 100.0 |

### Example 3

Ready-to-Eat Product—Bar with Vanilla Flavour

| Formula | % |
|---|---|
| Water | 82.2 |
| Carbohydrate including | 8.49 |
| pyruvate (10% of total) | |
| Protein (casein and soy 50/50) | 8.33 |
| Avicel, FMC CL611 | 0.46 |
| Vanilla Flavours | 0.4 |
| Butterscotch | 0.08 |
| Viscarin | 0.04 |

### Example 4

Production of Powder Formulation

A powder formulation consisting of the ingredients of table 1 were produced by mixing the ingredients and homogenising the powder.

TABLE 1

| | | Content in daily | Recommended daily |
|---|---|---|---|
| Content | Units | amount | amount |
| Energy | kcal | 810 | 800 |
| | KJ | 3400 | 3400 |
| Protein | g | 75 | |
| Soy protein | E % | 37 | |
| Carbohydrates | g | 96 | |
| | E % | 44 | |
| Fat | g | 15 | |
| | E % | 16 | |
| Linolic acid | g | 4.2 | 3.7 |
| Linolenic acid | g | 0.8 | 0.5 |
| unsaturated fatty acid | g | 7.0 | |
| Including MCT | g | 4.0 | |
| Fibres (barley) | g | 12 | |
| Vitamins | | | |
| Vitamin A | mg | 1.0 | 1-1.5 |
| Vitamin D | μg | 10 | 5-7.5 |
| Vitamin E | mg | 15 | 10-15 |
| Vitamin K | μg | 70 | 70-140 |
| Tiamin | mg | 1.2 | 1.4-2.1 |
| Riboflavin | mg | 1.6 | 1.7-2.6 |
| Niacin | mg | 18 | 18-27 |
| Vitamin B6 | mg | 1.5 | 2.1-3.2 |
| Vitamin B12 | μg | 2.4 | 3.0-4.5 |
| Folacin | μg | 400 | 200-300 |
| Biotin | μg | 100 | 100-200 |
| Pantotenic acid | mg | 5 | 4-7 |
| Vitamin C | mg | 90 | 60-90 |
| Minerals/salts | | | |
| Sodium (Na) | mg | 1685 | 1100-3300 |
| Potassium (K) | mg | 3100 | 1900-5600 |
| Calcium (Ca) | mg | 1200 | 800-1200 |
| Chloride (Cl) | mg | 1700 | 1700-5100 |
| Phosphor (P) | mg | 700 | 620-1860 |
| Magnesium (Mg) | mg | 350 | 350-525 |
| Iron (Fe) | mg | 22 | 15-23 |
| Zinc (Zn) | mg | 12 | 12-18 |
| Cupper (Cu) | mg | 2 | 2-3 |
| Iodine (I) | μg | 150 | 150-225 |
| Mangan (Mn) | mg | 2.5 | 2.5-5 |
| Chrom (Cr) | μg | 50 | 50-100 |
| Selen (Se) | μg | 55 | 50-100 |
| Molybden (Mo) | μg | 150 | 150-300 |
| Aromatics | mg | 5 | |
| Total | mg | 220 | |

### Example 5

Weight Loss with a Powder Formulation

41 subjects were allocated to weight reduction intervention with a powder formulation as described in Example 4, and allocated to either low or high physical activity during the intervention, the subjects were as described in Table 2.

TABLE 2
Anthropometry for all 41 subjects entering weight
reduction intervention with the formulation as
described in Example 4

| | A (n = 19) | B (n = 22) | *P-value |
|---|---|---|---|
| Age (years) | 42.4 ± 6.03 | 44.0 ± 8.4 | 0.475 |
| | (32-54) | (28-59) | |
| Height (m) | 1.67 ± 0.06 | 1.65 ± 0.07 | 0.488 |
| | (1.57-1.78) | (1.51-1.77) | |
| Weight (kg) | 90.94 ± 8.57 | 90.55 ± 11.53 | 0.903 |
| | (77.00-109.00) | (64.00-106.00) | |
| Fat free mass (kg) | 51.7 ± 3.2 | 51.7 ± 5.8 | 0.996 |
| | (46.2-61.0) | (37.8-63.6) | |
| Fat mass (kg) | 39.2 ± 6.6 | 38.9 ± 6.7 | 0.853 |
| | (28.6-48.6) | (26.2-48.8) | |
| Pct Fat | 43 ± 4 | 42 ± 3 | 0.876 |
| (% Body weight) | (36-48) | (35-48) | |
| Body mass | 32.8 ± 3.7 | 33.0 ± 3.3 | 0.817 |
| index (kg/m2) | (27.0-40.0) | (28.0-38.0) | |

Values are mean ± SD (min-max)
A: Subjects high in physical activity
B: Subjects low in physical activity
*Analyzed using One-way ANOVA

The intervention lasted for 7 weeks, and the changes due to intervention after 7 weeks, using the formulation as described in Example 4 as the only nutrient is listed in Table 3.

TABLE 3

| | A (n = 19) | B (n = 22) | *P-value |
|---|---|---|---|
| Weight (kg) | −6.75 (−7.89-−5.61)a | −6.73 (−7.82-−5.64)a | 0.984 |
| Fat free | −1.6 (−2.5-−0.7)b | −2.4 (−2.89-−1.86)a | 0.125 |
| mass (kg) | | | |
| Fat | −5.1 (−6.3-−4.0)a | −4.4 (−5.2-−3.5)a | 0.244 |
| mass (kg) | | | |
| Pct Fat | −3 (−4-−2)a | −2 (−2-−1)a | 0.665 |
| (% Body | | | |
| weight) | | | |
| Body mass | −2.7 (−3.7-−1.6)a | −2.3 (−2.7-−1.9)a | 0.134 |
| index | | | |
| (kg/m2) | | | |

Values are mean (95% confidence limits).
a: analyzed using paired t-test (p < 0.0001);
b: analyzed
using paired t-test (p = 0.002).
A: Subjects high in physical activity
B: Subjects low in physical activity
*Analyzed using One-way ANOVA

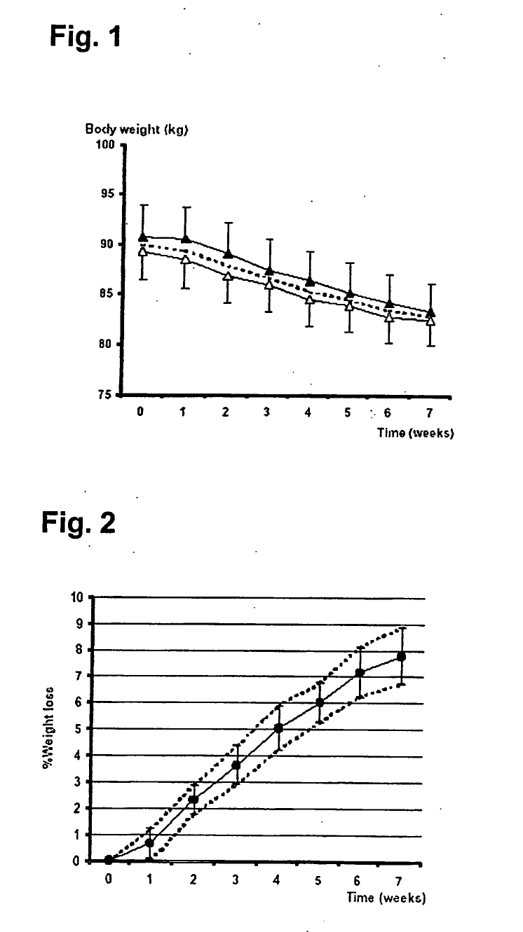

The results of weight loss are shown graphically in FIG. 1 for both groups and the percentage weight loss is shown graphically in FIG. 2.

The results show that a weight loss generated by using the powder formulation of Example 4 is found in both groups of high physical activity as well as low physical activity.

### Example 6

Weight Loss in Obese Patients with Knee Osteoarthritis

Obese patients with knee OA are encouraged to reduce their body weight. Because of the musculoskeletal pain associated with physical activity among obese patients with knee OA, most of these patients lead a sedentary life with little exercise. A dietary approach to diminish the weight will be beneficial for the patients in several aspects including a reduced load on the weight bearing joints.

Speasy® from NutriCare A/S, having the formulation of Example 4 provided the subjects with 3.4 MJ/day (810 kcal): 37 E % from protein, 47 E % from carbohydrate and 16 E % from fat.

29 obese patients (Body mass index (BMI)>28 kg/m²) with knee OA who were randomized to either the LED (women/men: 13/1) or a conventional (C) hypocaloric diet (5 MJ/day) (women/men: 12/3). Their age was 65.1±7.4 years (mean±SD) and 65.7±13.0 years, respectively, ranging from 45 to 90 years. The subjects' initial BMI was 36.3±4.9 kg/m² and 35.3±3.7 kg/m², and their lean body mass (LBM, assessed by electrical impedance) was 48.6±7.5 kg and 52.4±10.5 kg, respectively.

There was a significant weight reduction after both interventions. The LED intervention showed a reduction of 7.9 kg (95% confidence interval: 9.8 to 5.9; P<0.0001), whereas the C produced only 2.7 kg (3.9 to 1.5; P<0.001) (treatment difference P<0.0001). In the LED, the LBM decreased by 3.2 kg (3.9 to 2.5; P<0.0001) whereas the reduction was 1.9 kg (2.8 to 0.9; P<0.01) in C (treatment difference P<0.05). Thus, comparing the change in body composition (% LBM), we found a 1.1% (0.2 to 1.9; P<0.05) increase after LED, whereas no significant change after C: −0.4% (−1.3 to 0.4; P=0.27). 40.5% of weight loss in the LED group consisted of LBM versus 70% in the C group (P<0.02).

The LED showed a better weight reducing property, with a more favorable effect on body composition than a conventional hypocaloric diet after 8 weeks intervention. The results of this study demonstrate the possibility of reducing weight in elderly, sedentary patients, without loss of excessive lean body mass.

1-41. (canceled)

42. A low-energy-diet comprising as ingredients sources of
protein in an amount of from 50 g to 80 g per day,
carbohydrate in an amount of from 70 g to 120 g per day,
fat 5-20 g per day, including at least 3 g middle chain triglycerides per day,
and optionally minerals and pH-regulating agents, wherein one or more of the ingredient(s) has effect on at least two of the following mechanisms
the basal metabolic rate, and/or
the protein metabolism, and/or
the energy expenditure,
when the low-energy-diet is administered as the main or sole nutrition, and wherein the diet has an energy content in the range of from 600 kcal/d to 1200 kcal/d.

43. The low-energy-diet according to claim 42, wherein the diet has an energy content of approximately 800 kcal/d.

44. The low-energy-diet according to claim 42, wherein the source of protein is selected from casein, pork protein, and/or soy protein.

45. The low-energy-diet according to claim 44, wherein at least a portion of the source of protein is soy protein.

46. The low-energy-diet according to claim 42, wherein the diet comprises the protein source in an amount of from 60 g protein to 75 g protein.

47. The low-energy-diet according to claim 42, wherein the diet comprises the carbohydrate source in an amount of from 80 g carbohydrate to 110 g carbohydrate.

48. The low-energy-diet according to claim 42, wherein at least a part of the carbohydrate source is selected from dihydroxy acetone phosphate and/or pyruvate.

49. The low-energy-diet according to claim 42, wherein the medium chain triglycerides are selected from C6 triglycerides, C8 triglycerides and C10 triglycerides.

50. The low-energy-diet according to claim 42, wherein at least 3 g of the fat source is fish oil.

51. The low-energy-diet according to claim 42, wherein the diet comprises pH-regulating agent in an amount sufficient to reduce protein degradation.

52. The low-energy-diet according to claim 42, wherein the diet comprises bicarbonate in an amount of from 50 mmol/d to 70 mmol/d.

53. The low-energy-diet according to claim 42, wherein the diet comprises iron in an amount of from 20 mg/d to 30 mg/d.

54. The low-energy-diet according to claim 42, wherein the diet further comprises dietary fibers.

55. The low-energy-diet according to claim 54, wherein the dietary fibers are selected from barley fibers, sugar beet fibers and oat fibers.

56. The low-energy-diet according to claim 42, wherein the diet further comprises magnesium.

57. The low-energy-diet according to claim 56, wherein the magnesium is added as MgCl.

58. The low-energy-diet according to claim 42, wherein the diet is in the form of powder.

59. The low-energy-diet according to claim 58, wherein the powder is capable of being suspended or solved in liquid, such as liquid selected from water and milk.

60. The low-energy-diet according to claim 42, wherein the diet is in the form of ready-to-drink product.

61. The low-energy-diet according to claim 42, wherein the diet is in the form of bar product.

62. A method for treating overweight comprising administering to an individual in need thereof an effective amount of a low-energy-diet as defined in claim 42 as the main or sole nutrition daily.
